(19) United States
(12) Patent Application Publication
(10) Pub. No.: US 2017/0128575 A1
(43) Pub. Date: May 11, 2017

(54) LIQUID PHARMACEUTICAL FORMULATIONS OF PEMETREXED

(71) Applicant: DR. REDDY' S LABORATORIES LTD, Hyderabad (IN)

(72) Inventors: Sachin SHARMA, Kangra (IN); Amit Anil CHARKHA, Wardha (IN); Kumara Swamy DORNALA, Nalgonda (IN); Bhavesh Vallabhbhai PATEL, Amreli (IN); Harshal BHAGWATWAR, Hyderabad (IN)

(21) Appl. No.: 15/106,311

(22) PCT Filed: Dec. 19, 2014
(86) PCT No.: PCT/IB2014/067123

§ 371 Date: Jun. 19, 2016

(30) Foreign Application Priority Data
Dec. 19, 2013 (IN) 5951/CHE/2013

(51) Int. Cl. A61K 47/20; A61K 47/18; A61K 31/519

(52) U.S. Cl. CPC A61K 47/20; A61K 31/519; A61K 47/183

(57) ABSTRACT

The present invention provides stable liquid pharmaceutical formulation of pemetrexed or pharmaceutically acceptable salts thereof comprises at least one stabilizing agent selected from the group consisting of sodium formaldehyde sulfoxylate, EDTA and derivatives and mixtures thereof.

### INTRODUCTION

The present invention provides stable liquid pharmaceutical formulations of pemetrexed or pharmaceutically acceptable salts thereof comprising at least one stabilizing agent and processes for preparation thereof.

### BACKGROUND OF THE INVENTION

Pemetrexed, N-[4-[2-(2-amino-4,7-dihydro-4-oxo-1Hpyrrolo[2,3-d]pyrimidin-5-yl)ethyl]benzoyl]-L-Glutamic acid (also known as pemetrexed diacid), having the following formula:

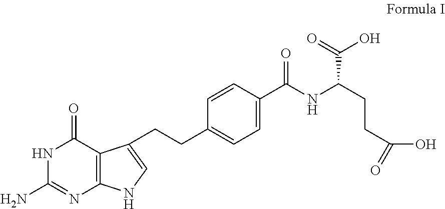 [SMILES: Nc1nc2[nH]cc(CCc3ccc(C(=O)N[C@@H](CCC(=O)O)C(=O)O)cc3)c2c(=O)[nH]1]

is a potent inhibitor of several folate-requiring enzymes and is useful for the treatment of non-small cell lung cancer and mesothelioma. Pemetrexed is available in the market under the brand name ALIMTA® with the active ingredient chemically described as L-Glutamic acid, N-[4-[2-(2-amino-4,7-dihydro-4-oxo-1H-pyrrolo[2,3-d]pyrimidin-5-yl)ethyl]benzoyl]-disodium salt heptahydrate (“Pemetrexed disodium heptahydrate”). The commercially-available product, ALIMTA®, is supplied as a sterile lyophilized powder available in single-use vials for intravenous infusion.

U.S. Pat. No. 5,344,932, which is incorporated herein by reference in its entirety, refers to pemetrexed diacid or pharmaceutically acceptable salts thereof and discloses parenteral administration of the same.

It is well known in the art that pemetrexed is prone for oxidative and hydrolytic degradations (Ref: Alimta®: European Public Assessment Report—Scientific Discussion; First published on Feb. 9, 2006 in official website of EMA). Pemetrexed is hydrolyzed both in acidic and basic conditions, resulting in decarboxylation of glutamic acid side chain. In presence of water and heat, same degradation products are formed, as in case of acidic and basic conditions. In presence of oxygen, two oxidative degradants are formed. Pemetrexed is also very prone for color change in aqueous solution formulation; color of the solution changes from colourless to yellow or green yellow. This color change may be attributed to oxidative degradation of pemetrexed.

It is reported that, pemetrexed liquid formulation was discontinued and a lyophilized formulation was introduced during clinical development of commercially available parenteral formulation of pemetrexed disodium (ALIMTA®) in order to address oxidative and hydrolytic degradation and considerable glass delamination problem (Ref: History of Regulatory Correspondence for ALIMTA® with USFDA available in USFDA website). Moreover, manufacturing process of such lyophilized product was developed to minimize oxidative degradation by reducing exposure of pemetrexed solution to oxygen and upon completion of lyophilization cycle the vacuum is neutralized with sterile filtered nitrogen, thus controlling the oxygen level in the drug product vial headspace. (Ref: Alimta®: European Public Assessment Report—Scientific Discussion; First published on Feb. 9, 2006 in official website of EMA). As on date liquid formulation of pemetrexed is not commercially available.

Lyophilization involves complex manufacturing processes, which in turn results in increasing manufacturing costs. Moreover, before administration to patients in clinics or hospitals, such lyophilized powder formulation must be reconstituted with suitable fluids and then diluted with pharmaceutically acceptable diluent to achieve desired concentration for administration. Therefore, an additional step of reconstitution is mandatory for such lyophilized formulation which causes inconveniences raising safety issues and risks of contamination by microorganisms. In addition, improper reconstitution may lead to high or low dosing to the patient in need.

In order to overcome inherent disadvantages of lyophilized formulation, some research group focused on discovering liquid pharmaceutical formulations of pemetrexed.

International application publication No. WO 2012/121523 describes method for preparing a pharmaceutical formulation in the form of solution for injection without antioxidant, the method of which comprises: (a) controlling a dissolved oxygen concentration in a solution for injection comprising pemetrexed or its salt to 1 ppm or less; and (b) filling a container for injection with the solution obtained from the step (a), in a closed system having an oxygen partial pressure of 0.2% v/v or less. WO'523 publication teaches various degassing methods to reduce dissolved oxygen level in finished formulation.

International application publication No. WO 2013/144814 describes a liquid pharmaceutical composition comprising pemetrexed or pharmaceutically acceptable salts thereof, wherein the composition is free from antioxidants, amino acids and chelating agents.

Although, WO'523 and WO'814 publications disclose process of preparation of liquid formulations free from antioxidants, large-scale preparation of such formulations involve difficult manufacturing processes which may raise stability concerns.

International application publication No. WO 2012/015810 describes liquid formulations comprising pemetrexed or a pharmaceutically acceptable salt thereof; an antioxidant such as lipoic acid, dihydrolipoic acid, methionine and mixtures thereof; a chelating agent such as lactobionic acid, sodium citrate, tri sodium citrate dihydrate and mixtures thereof; and optionally up to about 75% propylene glycol. The pH of the formulations is in a range of from about 8 to about 9.5.

Chinese Patent No. 101081305, again discloses a liquid formulation of pemetrexed comprising antioxidants like L-arginine, L-glutathione, L-methionine and L-tryptophan.

International application publication No. WO 2013/165130 describes a liquid formulations of pemetrexed or pharmaceutically acceptable salts thereof comprising pemetrexed at a concentration form 1 to 100 mg/ml and at least one of stabilizer selected from, 0.4×10 −6 M to 0.1×10 −4 M sodium sulfide or 0.2×10 −6 M to 0.8×10 −5 M sodium sulfite.

U.S. Pat. No. 6,686,365 teaches that a simple, isotonic saline solution of pemetrexed is not pharmaceutically acceptable for commercial purposes due to degradation of pemetrexed in solution forming unacceptable impurities. Therefore, the inventors of US'365 patent provided liquid parenteral formulations comprising pemetrexed disodium, at least one antioxidant selected from the group consisting of monothioglycerol, L-cysteine, and thioglycolic acid, and a pharmaceutically acceptable excipient. However, as mentioned in WO'810 publication, practical experiments performed according to the examples of US'365 patent, wherein formulations containing pemetrexed disodium at a concentration of 25 mg/ml, one of mortothioglycerol, L-cysteine or thioglycolic acid at recommended concentrations of 2.4 mg/ml and water for injection failed to demonstrate desirable long term stability during shelf life. The pemetrexed content dropped down well below acceptable levels.

Moreover, US' 365 patent also teaches that, common antioxidants, such as sodium formaldehyde sulfoxylate, sodium metabisulfite, ascorbic acid, sodium EDTA, monoethanolamine gentisate, sodium bisulfite, do not provide the desired formulation characteristics.

Surprisingly, inventors of the present application discovered that a liquid formulation of pemetrexed can be prepared using “common antioxidants”, even with those that are reported as “non-workable” in the specification of US'365 patent. Such formulations comprise pemetrexed or pharmaceutically acceptable salts thereof and at least one stabilizing agent selected from the group consisting of sodium formaldehyde sulfoxylate, EDTA and derivatives and mixtures thereof.

### SUMMARY OF THE INVENTION

Aspects of the present invention provide stable liquid pharmaceutical formulations of pemetrexed or pharmaceutically acceptable salts thereof comprising at least one stabilizing agent.

Another aspect of the invention provides simple processes for preparation of such stable liquid formulations of pemetrexed and methods of using such formulations for treating various types of cancers in mammals.

In embodiments, stable liquid pharmaceutical formulation of pemetrexed or pharmaceutically acceptable salts thereof comprises at least one stabilizing agent selected from the group of antioxidant, chelating agent, preservative or suitable mixtures thereof.

In embodiments, stable liquid pharmaceutical formulation of pemetrexed or pharmaceutically acceptable salts thereof comprises at least one stabilizing agent selected from the group consisting of sodium formaldehyde sulfoxylate, EDTA and derivatives and mixtures thereof.

In embodiments, stable liquid pharmaceutical formulation comprises from about 2% to about 80% or about 15% to about 60% or about 20% to about 50% of pemetrexed or pharmaceutically acceptable salts thereof.

In embodiments, stable liquid pharmaceutical formulation of pemetrexed comprises stabilizing agent at a concentration from about 0.001 mg/ml to about 10 mg/ml or from about 0.01 mg/ml to about 5 mg/ml.

In embodiments, stable liquid pharmaceutical formulation of pemetrexed comprises sodium formaldehyde sulfoxylate at a concentration from about 0.01 mg/ml to about 3 mg/ml.

In embodiments, stable liquid pharmaceutical formulation of pemetrexed comprises EDTA or derivative thereof at a concentration from about 0.01 mg/ml to about 5 mg/ml.

In embodiments, stable liquid pharmaceutical formulation comprising pemetrexed are in the form of ready-to-use solutions or concentrates for further dilution.

In embodiments, stable liquid pharmaceutical formulation of pemetrexed comprises aqueous solvent, non-aqueous solvent or suitable mixture thereof.

In embodiments, stable liquid pharmaceutical formulation of pemetrexed comprising of at least one stabilizing agent and aqueous solvent.

In embodiments, stable liquid pharmaceutical formulation of pemetrexed comprising of at least one stabilizing agent and non-aqueous solvent.

In embodiments, stable liquid pharmaceutical formulation of pemetrexed has a pH from about pH 6 to about 8.5.

In embodiments, stable liquid pharmaceutical formulation of pemetrexed comprises not more than about 3% of total impurities, more preferably not more than about 2% of total impurities, expressed as percentages of the labeled pemetrexed content.

In embodiments, stable liquid pharmaceutical formulation of pemetrexed comprises not more than about 0.5% of maximum individual unknown impurity, more preferably not more than about 0.2% of maximum individual unknown impurity, expressed as percentages of the labeled pemetrexed content.

In embodiments, stable liquid pharmaceutical formulation of pemetrexed comprises not more than about 2.0% of impurity F, more preferably not more than about 1.0% of impurity F, expressed as percentages of the labeled pemetrexed content.

In embodiments, stable liquid pharmaceutical formulation of pemetrexed comprises not more than about 3% of total impurities and not more than about 0.5% of maximum individual unknown impurity and not more than about 2.0% of impurity F, expressed as percentages of the labeled pemetrexed content.

### DETAILED DESCRIPTION

Aspects of the present application relate to stable liquid pharmaceutical formulations of pemetrexed or pharmaceutically acceptable salts thereof comprising at least one stabilizing agent. Another aspect of the invention provides processes for preparation of such liquid formulations comprising pemetrexed and methods of using such formulations for treating various types of cancers in mammals.

In embodiments, the present application provides liquid formulation comprising pemetrexed at concentrations about 1 mg/mL to about 100 mg/mL. In embodiments, the concentrations of pemetrexed pharmaceutically acceptable salt, equivalent to pemetrexed are in the range of about 5 mg/mL to about 80 mg/mL, or about 15 mg/mL to about 60 mg/mL or about 20 mg/mL to about 50 mg/mL. In one preferred embodiment, the concentration of pemetrexed pharmaceutically acceptable salt, equivalent to pemetrexed is 40 mg/mL. In another preferred embodiment, the concentration of pemetrexed pharmaceutically acceptable salt, equivalent to pemetrexed is 25 mg/mL.

The term “pemetrexed” includes the compound pemetrexed, pharmaceutically acceptable salts of pemetrexed, isomers, solvates, prodrugs, complexes and hydrates, anhydrous forms thereof, and any polymorphic or amorphous forms or combinations thereof.

As used herein, the term “pharmaceutically acceptable salts” includes pemetrexed salts with bases, such as, those formed from the alkali metals, alkaline earth metals, non-toxic metals, and mono-, di- and trisubstituted amines, for example the sodium, potassium, lithium, calcium, magnesium, aluminum, zinc, ammonium, trimethylammonium, triethanolammonium, pyridinium, and substituted pyridinium salts. In one embodiment, “pharmaceutically acceptable salt” is pemetrexed ditromethamine. In another embodiment, “pharmaceutically acceptable salt” is pemetrexed disodium. In another embodiment, “pharmaceutically acceptable salt” is pemetrexed dipotassium.

The term “stable formulation” refers to any preparation of pemetrexed or pharmaceutically acceptable salts thereof having sufficient physical and chemical stability to allow storage at a convenient temperature, such as between about 0° C. and about 50° C., for a commercially reasonable period of time. The term “physical stability” refers to maintenance of color, dissolved oxygen level, head space oxygen level, and particulate matter and the term “chemical stability” relates to formation of drug-related impurities in terms of total impurity, maximum individual unknown impurity and single maximum individual impurity. For the purpose of the present invention chemical stability also includes stabilizing agent content and maintenance of pH of the finished formulation. For pharmaceutical products, stability is required for commercially relevant times after manufacturing, such as for about 6, 12, 18, 24 or 36 months, during which a product is kept in its original packaging under specified storage condition.

For the purpose of this invention stable liquid pharmaceutical formulation of pemetrexed comprise not more than about 3% of total impurities, expressed as percentages of the labeled pemetrexed content.

In embodiments, stable liquid pharmaceutical formulation of pemetrexed comprises not more than about 3% of total impurities, more preferably not more than about 2% of total impurities, expressed as percentages of the labeled pemetrexed content.

In embodiments, stable liquid pharmaceutical formulation of pemetrexed comprises not more than about 0.5% of maximum individual unknown impurity, more preferably not more than about 0.2% of maximum individual unknown impurity, expressed as percentages of the labeled pemetrexed content.

In embodiments, stable liquid pharmaceutical formulation of pemetrexed comprises not more than about 2.0% of impurity F, more preferably not more than about 1.0% of impurity F, expressed as percentages of the labeled pemetrexed content.

In embodiments, stable liquid pharmaceutical formulation of pemetrexed comprises not more than about 3% of total impurities and not more than about 0.5% of maximum individual unknown impurity and not more than about 2.0% of impurity F, expressed as percentages of the labeled pemetrexed content.

Details of Impurity F:

| Impu- | Chemical | | RR | RR |
|---|---|---|---|---|
| rity | Name | Structure | T | F |
| Imp F | 2,2′-((4,4′- ((methylenebis(2- amino-4-oxo-4,5- dihydro-1H- pyrrolo[2,3- d]pyrimidine-5,5- diyl))bis(ethane- 2,1- diyl))bis(benzoyl)) bis(azanediyl)) diglutaric acid | 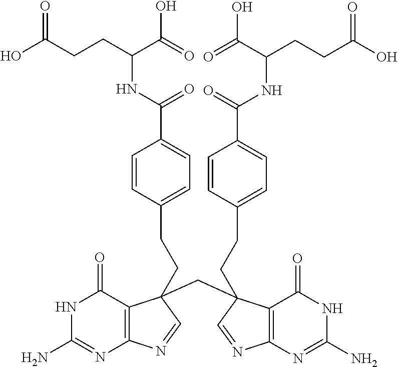 [SMILES: Nc1nc2c(c(=O)[nH]1)C(CCc1ccc(C(=O)NC(CCC(=O)O)C(=O)O)cc1)(CC1(CCc3ccc(C(=O)NC(CCC(=O)O)C(=O)O)cc3)C=Nc3nc(N)[nH]c(=O)c31)C=N2] | 1.15 | 0.78 |

As used herein liquid formulation includes, but not limited to, liquid form ready for injection or concentrate form for further dilution before injection or infusion.

In one embodiment, the stable liquid pharmaceutical formulation of pemetrexed is presented as liquid form ready for injection.

In another embodiment, the stable liquid pharmaceutical formulation of pemetrexed is presented as concentrate form for further dilution before injection or infusion.

The stable liquid formulation of the invention comprises pemetrexed or pharmaceutically acceptable salts thereof and at least one stabilizing agent.

The stable liquid formulation for the purpose of this invention comprises stabilizer(s) which include antioxidant, chelating agent, preservative or suitable mixtures thereof. Examples of stabilizers include, but not limited to, sodium formaldehyde sulfoxylate, sodium hydrogen sulfite, ascorbic acid and derivatives, EDTA and derivatives, monoethanolamine gentisate, butylated hydroxy anisole, butylated hydroxy toluene, glutathione, propionic acid, propyl gallate, tocopherols and derivatives, acetone sodium bisulfite, sodium dithionite, citric acid and derivatives, lactobionic acid, tribasic (tri sodium citrate dihydrate) or suitable mixtures thereof.

In one embodiment, the stable liquid pharmaceutical formulation of pemetrexed comprises stabilizing agent selected from the group consisting of sodium formaldehyde sulfoxylate, EDTA and derivatives and mixtures thereof.

As per the common general knowledge, the shelf stability increases with increasing concentrations of stabilizing agent. However, it was surprisingly found that lowering the concentration of stabilizing agent increases the shelf stability. Accordingly, in one embodiment, the invention further relates to the unexpected discovery of effective concentration of stabilizing agent.

In embodiments, the stable liquid pharmaceutical formulation of pemetrexed comprises sodium formaldehyde sulfoxylate or hydrates thereof. For the purpose of this invention the term “sodium formaldehyde sulfoxylate” includes the compound sodium formaldehyde sulfoxylate, pharmaceutically acceptable salts thereof, isomers, solvates, prodrugs, complexes and hydrates, anhydrous forms thereof or combinations thereof.

As per one embodiment, concentration of sodium formaldehyde sulfoxylate ranges from about 0.001 mg/ml to about 5 mg/ml. As per one more preferred embodiment, concentration of sodium formaldehyde sulfoxylate ranges from about 0.01 mg/ml to about 3 mg/ml. In another embodiment, the concentration of sodium formaldehyde sulfoxylate is about 2 mg/ml or 1 mg/ml or 0.75 mg/ml or about 0.25 mg/ml or about 0.2 mg/ml or about 0.1 mg/ml or about 0.05 mg/ml or about 0.01 mg/ml or about 0.001 mg/ml.

In one embodiment, the stable liquid pharmaceutical formulation of pemetrexed comprises EDTA and derivatives thereof. As per one preferred embodiment, concentration of EDTA ranges from about 0.001 mg/ml to about 10 mg/ml. As per one more preferred embodiment, concentration of EDTA ranges from about 0.01 mg/ml to about 5 mg/ml.

In one embodiment, the stable liquid pharmaceutical formulation of the present invention comprises pemetrexed disodium and sodium formaldehyde sulfoxylate.

In one embodiment, the stable liquid pharmaceutical formulation of the present invention comprises pemetrexed disodium equivalent to pemetrexed of about 10 mg/mL to about 80 mg/mL and sodium formaldehyde sulfoxylate.

In one embodiment, the stable liquid pharmaceutical formulation of the present invention comprises pemetrexed disodium equivalent to pemetrexed of about 10 mg/mL to about 80 mg/mL and sodium formaldehyde sulfoxylate from about 0.01 mg/ml to about 5 mg/ml.

In one embodiment, the stable liquid pharmaceutical formulation of the present invention comprises pemetrexed disodium equivalent to pemetrexed of about 10 mg/mL to about 80 mg/mL, sodium formaldehyde sulfoxylate from about 0.01 mg/ml to about 5 mg/ml and disodium EDTA from about 0.01 mg/ml to about 5 mg/ml.

In one embodiment, the stable liquid pharmaceutical formulation of the present invention comprises pemetrexed ditromethamine and sodium formaldehyde sulfoxylate.

In one embodiment, the stable liquid pharmaceutical formulation of the present invention comprises pemetrexed ditromethamine equivalent to pemetrexed of about 10 mg/mL to about 80 mg/mL and sodium formaldehyde sulfoxylate from about 0.01 mg/ml to about 5 mg/ml.

In one embodiment, the stable liquid pharmaceutical formulation of the present invention comprises pemetrexed ditromethamine equivalent to pemetrexed of about 10 mg/mL to about 80 mg/mL and sodium formaldehyde sulfoxylate from about 0.01 mg/ml to about 5 mg/ml.

In one embodiment, the stable liquid pharmaceutical formulation of the present invention comprises pemetrexed ditromethamine equivalent to pemetrexed of about 10 mg/mL to about 80 mg/mL, sodium formaldehyde sulfoxylate from about 0.01 mg/ml to about 5 mg/ml and disodium EDTA from about 0.01 mg/ml to about 5 mg/ml.

As described herein, a stable liquid pharmaceutical formulation of pemetrexed is achieved by dissolving pemetrexed and stabilizing agent of this invention in suitable solvent(s).

Suitable solvents comprise aqueous solvent, non-aqueous solvent or suitable mixture thereof.

Suitable non-aqueous solvents comprise, but not limited to, ethanol, propylene glycol, polyethylene glycols, glycerin, benzyl alcohol, glycofurol, solketal, ethyl lactate, tetrahydrofurfuryl alcohol, glycerol formal, ethylene glycol, butylene glycol, polypropylene glycol, polybutylene glycol, polysorbates, niacinamide, formic acid, n-butanol, isopropanol (IPA), acetic acid, methanol, polyvinylpyrrolidone (PVP), methoxypropylene glycol (MPEG) or suitable mixtures thereof.

In one embodiment, suitable solvent is water. In another embodiment, suitable solvent comprise water and non-aqueous solvent selected form ethanol, propylene glycol, polyethylene glycols, glycerin, and benzyl alcohol.

This liquid formulation may additionally comprise at least one pharmaceutically acceptable excipient, for example, but not limited to, tonicity contributing agent, solubilizers, buffers, pH adjusting agent or suitable mixtures thereof.

Suitable tonicity contributors include, but not limited to, dextrose, sodium chloride, polyhydroxy compounds such as sugars, mono, di- or polysaccharides for example mannitol, lactose, sucrose, maltose, dextrose, dextran, trehalose and hetastarch, polyalcohols and their derivatives, for example glycerin or mixtures thereof. Suitable tonicity contributor concentration in the formulations will be from about 0.1 mg/ml to about 100 mg/ml.

Suitable buffers include, but not limited to, acetate, citrate, glutamate, phosphate, benzoate, lactate, ascorbate, tartarate, succinate, glycine, triethanolamine, diethanolamine, tromethamine or suitable mixture thereof.

Suitable pH adjusting agents include, but not limited to, sodium hydroxide solution, hydrochloric acid solution or tromethamine solution.

The pH of the liquid pemetrexed formulation will vary from about 6 to about 8.5. More preferably, it varies from about pH 6.5 to about 8. Most preferably, it varies from about 6.6 to about 7.8. Standard modifications of the formulation can provide formulations of various pH within the contemplation of this invention.

As described herein, a stable liquid pharmaceutical formulation of pemetrexed is prepared by mixing pemetrexed, stabilizing agent and optionally other pharmaceutically acceptable excipients in suitable solvent system.

In one embodiment, the formulation is prepared by a process comprising steps of:

(1) Purging nitrogen to reduce dissolved oxygen of solvent system below 2 ppm.

(2) Dissolving pemetrexed in solvent system.

(3) Dissolving stabilizing agent(s) in solvent system.

(4) Adjusting the pH of the solution from about pH 6 to about 8.5.

(5) Sterilizing using 0.22 μm sterile filters.

(6) Filling and sealing in suitable vial.

In one preferred embodiment, the formulation is prepared by a process comprising steps of:
(1) Purging nitrogen to reduce dissolved oxygen of solvent system below 2 ppm.
(2) Dissolving pemetrexed in solvent system.
(3) Dissolving stabilizing agent, selected from the group consisting of sodium formaldehyde sulfoxylate, disodium edetate, and mixtures thereof, in solvent system.
(4) Adjusting the pH of the solution from about pH 6 to about 8.5.
(5) Sterilizing using 0.22 μm sterile filters.
(6) Filling and sealing in suitable vial.

In another preferred embodiment, the formulation is prepared by a process comprising steps of:
(1) Purging nitrogen to reduce dissolved oxygen of solvent system below 2 ppm.
(2) Dissolving pemetrexed at a concentration from about 20 mg/ml to about 50 mg/ml in solvent system.
(3) Dissolving sodium formaldehyde sulfoxylate at a concentration from about 0.01 mg/ml to about 5 mg/ml in solvent system.
(4) Adjusting the pH of the solution from about pH 6 to about 8.5.
(5) Sterilizing using 0.22 μm sterile filters.
(6) Filling and sealing in suitable vial.

In another preferred embodiment, the formulation is prepared by a process comprising steps of:
(1) Purging nitrogen to reduce dissolved oxygen of solvent system below 2 ppm.
(2) Dissolving pemetrexed at a concentration from about 20 mg/ml to about 50 mg/ml in solvent system at processing temperature in a range from about 2° C. to about 8° C.
(3) Dissolving sodium formaldehyde sulfoxylate at a concentration from about 0.01 mg/ml to about 5 mg/ml in solvent system at processing temperature in a range from about 2° C. to about 8° C.
(4) Adjusting the pH of the solution from about pH 6.5 to about 8.
(5) Sterilizing using 0.22 μm sterile filters.
(6) Filling and sealing in suitable vial.

In another preferred embodiment, the formulation is prepared by a process comprising steps of:
(1) Purging nitrogen to reduce dissolved oxygen of solvent system below 2 ppm.
(2) Dissolving mannitol in solvent system.
(3) Dissolving pemetrexed at a concentration from about 20 mg/ml to about 50 mg/ml in water at processing temperature in a range from about 2° C. to about 8° C.
(4) Dissolving sodium formaldehyde sulfoxylate at a concentration from about 0.01 mg/ml to about 5 mg/ml in water at processing temperature in a range from about 2° C. to about 8° C.
(4) Adjusting the pH of the solution from about pH 6.6 to about 7.8.
(5) Sterilizing using 0.22 μm sterile filters.
(6) Filling and sealing in suitable vial.

For the purpose of this invention, dissolved oxygen content is reduced by purging and blanketing drug solution during manufacturing and hold respectively with inert gas. Nitrogen is most preferred inert gas for reducing dissolved oxygen. Head space oxygen is controlled through flushing air inside the vials with inert gas before and after filling of prepared formulation and immediately closing vial with rubber stopper. Alternatively, prepared formulation filled vials are partially stoppered with slotted stopper and then loaded in lyophilizer. In lyophilizer, vacuum is applied which is released by inert gas. Vials are then completely stoppered, preferably inside lyophilizer in inert gas environment. Nitrogen is most preferred inert gas for reducing head space oxygen.

In one embodiment, stable liquid pharmaceutical formulation of pemetrexed is prepared by first dissolving stabilizing agent(s) in solvent system, followed by dissolving pemetrexed. In another embodiment, stable liquid pharmaceutical formulation of pemetrexed is prepared by first dissolving pemetrexed in solvent system, followed by dissolving stabilizing agent(s). Order of addition of drug and excipients does not matter for the purpose of the present invention.

The liquid formulation of pemetrexed can be analyzed by common techniques, such as high performance liquid chromatography, to determine their drug content and the concentrations of drug-related impurities.

The pemetrexed formulations of the present invention can be packaged in any suitable sterile vial or container fit for the sterile storage of a pharmaceutical such as pemetrexed for extended periods of time. Suitable containers can be glass vials, i.e. treated vials, molded glass vials, and CZ resin vials, polypropylene or polyethylene vials or other special purpose containers. Containers are of a size sufficient to hold one or more doses of pemetrexed. The pemetrexed formulations can also be stored in vials which are designed to minimize delamination and pitting problems, for example, PICVD (Plasma Impulse Chemical Vapor Deposition) and the like.

The pharmaceutical formulation of the present invention is resistant to color changes, degradation and ensures acceptable shelf life. The claimed formulations can be diluted to the desired administration concentration or administered directly by the health care provider.

The stable liquid pharmaceutical formulation of pemetrexed of the present invention can be diluted with 0.9% Sodium Chloride Injection (preservative free) solution and appropriate quantity of such diluted solution may be administered to a patient in need.

In another aspect of the invention provides methods of treating a pemetrexed sensitive disease in mammals. Pemetrexed sensitive diseases include, but are not limited to, cancers, such as malignant pleural mesothelioma and non-small cell lung cancer. The methods include administering an effective amount of a pemetrexed containing composition as described herein to a mammal in need thereof.

Certain specific aspects and embodiments of the present application will be explained in more detail with reference to the following examples, which are provided only for purposes of illustration and should not be construed as limiting the scope of the present application in any manner.

### EXAMPLES

### Example 1

(Qty/mL)

| | | | | | | | | | | |
|---|---|---|---|---|---|---|---|---|---|---|
| Pemetrexed | 40 mg | 40 mg | 40 mg | 40 mg | 40 mg | 40 mg | 40 mg | 40 mg | 40 mg | 40 mg |
| disodium | | | | | | | | | | |
| equivalent to | | | | | | | | | | |
| Pemetrexed | | | | | | | | | | |
| Antioxidant | Sodium | Sodium | L- | L- | L- | Thiodipropionic | Phenyl | Glycine | Sodium | Trisodium |
| | meta- | formaldehyde | Methionine | Arginine | Tryptophan | acid | alanine | | sulfite | citrate |
| | bisulfite | sulfoxylate | | | | | | | | dihydrate |
| Antioxidant | 0.3 mg | 1 mg | 1 mg | 1 mg | 1 mg | 1 mg | 1 mg | 1 mg | 1 mg | 1 mg |
| concen- | | | | | | | | | | |
| tration | | | | | | | | | | |
| Description | Clear | Clear | Clear | Clear | Clear | Clear | Clear | Clear | Clear | Clear |
| of solution - | colorless | colorless | colorless | colorless | colorless | colorless | colorless | colorless | colorless | colorless |
| Initial | | | | | | | | | | |
| Description | Very | Clear | Light | Light | Light | Light | Light | Light | Light | Light |
| after | Light | colorless | yellow | yellow | yellow | yellow | yellow | yellow | yellow | yellow |
| 15 min | yellow | | | | | | | | | |
| autoclave | | | | | | | | | | |
| Description | Light | Clear | Light | Light | Light | Light | Light | Light | Light | Light |
| after | yellow | colorless | yellow | yellow | yellow | yellow | yellow | yellow | yellow | yellow |
| 2 hrs | | | | | | | | | | |
| autoclave | | | | | | | | | | |

Manufacturing Process:

1. Stabilizing agent (Sodium metabisulfite, Sodium formaldehyde sulfoxylate, L-Methionine, L-Arginine, L-Tryptophan, Thiodipropionic acid, Phenyl alanine, Glycine, Sodium sulfite, EDTA, Trisodium citrate dihydrate) is added to 90% of required Water for injection and stirred till clear solution is obtained. Dissolved oxygen level is maintained below 2 ppm during bulk solution manufacturing.

2. Pemetrexed disodium is added to the solution of step 1 and stirred till clear solution is obtained.

3. pH of the solution of step 2 is adjusted to 6.5 to 7.5 using only hydrochloric acid solution, and volume is made up with remaining water.

4. Solution of step 3 is purged with nitrogen and subsequently filtered through 0.22 μm sterile filters. Solution is stored under nitrogen blanket until filling is initiated.

5. The solution of step 4 is dispensed into depyrogenated USP type-I glass vials and stoppered in presence of nitrogen and sealed.

6. The solution was autoclaved for specified time under pre-defined condition.

7. The contents of the initial and autoclaved vials are analyzed for any color change. Data provided in above table.

From the above data, it is evident that sodium formaldehyde sulfoxylate is providing clear colourless solution in stress study, whereas other antioxidants are not providing clear colourless solution, hence are not suitable.

### Example 2-5

| Ingredients | 2 | 3 | 4 | 5 |
|---|---|---|---|---|
| Pemetrexed disodium equivalent | 40 | 40 | 40 | 40 |
| to Pemetrexed (mg/ml) | | | | |
| Mannitol (mg/ml) | — | — | 25 | 25 |
| Sodium formaldehyde sulfoxylate | 0.75 | 0.25 | 0.2 | 0.1 |
| (mg/ml) | | | | |
| Water for Injection | 1 | 1 | 1 | 1 |
| | ml | ml | ml | ml |

Manufacturing Process:

1. Sodium formaldehyde sulfoxylate is added to 90% of required Water for injection and stirred till clear solution is obtained. Dissolved oxygen level is maintained below 2 ppm during bulk solution manufacturing.

2. Mannitol (for example 4-5), is added to the solution of step 1 under stirring and stirred till clear solution is obtained.

3. Pemetrexed disodium is added to the solution from above step and stirred till clear solution is obtained.

4. pH of the solution of step 3 is adjusted to specific value using hydrochloric acid solution or sodium hydroxide solution and volume is made up with remaining water for injection.

5. Solution of step 4 is purged with nitrogen and subsequently filtered through 0.22 μm sterile filters. Solution is stored under nitrogen blanket until filling is initiated.

6. The solution of step 5 is dispensed into depyrogenated USP type-I glass vials and stoppered in presence of nitrogen and sealed. Head space oxygen in vials is maintained below 5%.

7. The solution filled vials are heat sterilized at 121° C.

8. Vials are stored at 25±2° C. and 60% relative humidity (RH) for 3 months, or at 40±2° C. and 75% RH, for 3 months.

9. The contents of the initial and stored vials are analyzed for impurity content using suitable HPLC method. Data provided in below table.

| Time | Impurity | 2 | 3 | 4 | 5 |
|---|---|---|---|---|---|
| Initial (%) | TI | 1.74 | 1.04 | 0.31 | 0.22 |
| 3 Month 25° C./60% RH (%) | TI | — | — | 1.43 | 0.96 |
| 3 Month at 40° C./75% RH (%) | TI | — | — | 2.21 | 1.47 |

*TI-Total Impurities

### Example 6-11

| Ingredients | 6 | 7 | 8 | 9 | 10 | 11 |
|---|---|---|---|---|---|---|
| Pemetrexed disodium | 40 | 40 | 40 | 40 | 40 | 40 |
| equivalent to Pemetrexed | | | | | | |
| (mg/ml) | | | | | | |
| Mannitol(mg/ml) | 25 | 25 | 25 | 25 | 25 | 25 |
| Sodium formaldehyde | 0.05 | 0.04 | 0.03 | 0.02 | 0.01 | 0.00 |
| sulfoxylate (mg/ml) | | | | | | |
| Water for Injection | 1 ml | 1 ml | 1 ml | 1 ml | 1 ml | 1 ml |

Manufacturing Process:

1. Mannitol is added to 90% of required Water for injection and stirred till clear solution is obtained. Dissolved oxygen level is maintained below 2 ppm during bulk solution manufacturing.

2. Pemetrexed disodium is added to the solution from above step and stirred till clear solution is obtained.

3. pH of the solution of step 3 is adjusted to specific value using hydrochloric acid solution or sodium hydroxide solution 4. Sodium formaldehyde sulfoxylate is added to the solution and stirred till clear solution is obtained.

5. Volume is made up with remaining water for injection.

6. Solution of step 5 is purged with nitrogen and subsequently filtered through 0.22 μm sterile filters. Solution is stored under nitrogen blanket until filling is initiated.

7. The solution of step 6 is dispensed into depyrogenated USP type-I glass vials and stoppered in presence of nitrogen and sealed.

8. The solution is purged with nitrogen and sterile filtered. The formulation is dispensed into vials and then stoppered with Bromobutyl stopper. Caps are attached using a crimper. Head space oxygen in vials is maintained below 5%.

9. The solution filled vials are heat sterilized at 121° C.

10. Vials are stored at 60° C. for 8 days.

11. The contents of the stored vials are analyzed for any color change and impurity content using suitable HPLC method. Data provided in below table.

| Time | Parameter | 6 | 7 | 8 | 9 | 10 | 11 |
|---|---|---|---|---|---|---|---|
| Initial | Appearance | Clear | Clear | Clear | Clear | Clear | Clear |
| | | with | with | with | with | with | with |
| | | very | very | very | very | very | very |
| | | mild | mild | mild | mild | mild | mild |
| | | yellow | yellow | yellow | yellow | yellow | yellow |
| | | tinge | tinge | tinge | tinge | tinge | tinge |
| 8 days | Appearance | Clear | Clear | Clear | Clear | Clear | Clear |
| after | | solution | solution | solution | solution | solution | with |
| storage at | | | | | | | mild |
| 2-8° C. | | | | | | | yellow |
| | | | | | | | tinge |
| 8 days | Appearance after | Clear | Clear | Nearly | Clear | Clear | Yellow |
| after | storage at 60° C. for | solution | solution | clear | with | with | color |
| storage at | 8 days | | | | very | very | |
| 60° C. | | | | | mild | mild | |
| | | | | | yellow | yellow | |
| | | | | | tinge | tinge | |
| | | | | | like | like | |
| | | | | | initial | initial | |
| | TI | 1.464 | 1.474 | 1.244 | 1.224 | 1.079 | 1.025 |
| | MIUI | 0.226 | 0.246 | 0.238 | 0.235 | 0.227 | 0.274 |
| | Imp F | 0.672 | 0.571 | 0.437 | 0.30 | 0.165 | 0.019 |

TI—Total Impurities
MIUI—Maximum individual unknown impurity

From above data, it is evident that sodium formaldehyde sulfoxylate provides stable liquid pharmaceutical formulation of pemetrexed or pharmaceutically acceptable salt thereof. It was also observed that lower concentrations of sodium formaldehyde sulfoxylate provide lower amount of impurity.

### Example 12-15

Composition is Same as Example 6.

Manufacturing Process for Example 12:

1. 90% of required Water for injection is taken in manufacturing vessel. Dissolved oxygen level is maintained below 2 ppm during bulk solution manufacturing.

2. Mannitol is added to the solution of step 1 at processing temperature of 25° C. and stirred till clear solution is obtained.

3. Pemetrexed disodium is added to the solution from above step and stirred till clear solution is obtained.

4. Sodium formaldehyde sulfoxylate is added to the solution of step 3 and stirred till clear solution is obtained.

5. pH of the solution of step 4 is adjusted to specific value using hydrochloric acid solution or sodium hydroxide solution

6. Solution was cooled to 2° C. to 8° C.

7. Volume is made up with remaining water for injection.

8. Solution of step 7 is purged with nitrogen and subsequently filtered through 0.22 μm PVDF sterile filters.

9. This bulk solution is analyzed for impurity content using suitable HPLC method.

Manufacturing Process for Example 13:

90% of required Water for injection is taken in manufacturing vessel. Dissolved oxygen level is maintained below 2 ppm during bulk solution manufacturing.

1. Sodium formaldehyde sulfoxylate is added to the solution of step 1 processing temperature of 61° C. and stirred till clear solution is obtained.

2. Solution was cooled to 25° C. and mannitol is added and stirred till clear solution is obtained.

3. Pemetrexed disodium is added to the solution from above step and stirred till clear solution is obtained.

4. pH of the solution of step 4 is adjusted to specific value using hydrochloric acid solution or sodium hydroxide solution.

5. Solution was cooled to 2° C. to 8° C.

6. Volume is made up with remaining water for injection.

7. Solution of step 7 is purged with nitrogen and subsequently filtered through 0.22 μm PVDF sterile filters.

8. This bulk solution is analyzed for impurity content using suitable HPLC method.

Manufacturing Process for Example 14:

1. 90% of required Water for injection is taken in manufacturing vessel. Dissolved oxygen level is maintained below 2 ppm during bulk solution manufacturing.

2. Sodium formaldehyde sulfoxylate is added to the solution of step 1 processing temperature of 25° C. and stirred till clear solution is obtained.

3. Mannitol is added and stirred till clear solution is obtained.

4. Pemetrexed disodium is added to the solution from above step and stirred till clear solution is obtained.

5. pH of the solution of step 4 is adjusted to specific value using hydrochloric acid solution or sodium hydroxide solution.

6. Solution was cooled to 2° C. to 8° C.

7. Volume is made up with remaining water for injection.

8. Solution of step 7 is purged with nitrogen and subsequently filtered through 0.22 μm PVDF sterile filters.

9. This bulk solution is analyzed for impurity content using suitable HPLC method.

Manufacturing Process for Example 15:

1. 90% of required Water for injection is taken in manufacturing vessel. Dissolved oxygen level is maintained below 2 ppm during bulk solution manufacturing.

2. Mannitol is added to the solution of step 1 and stirred till clear solution is obtained.

3. Solution was cooled to 2° C. to 8° C.

4. Pemetrexed disodium is added to the solution from above step and stirred till clear solution is obtained.

5. pH of the solution of step 4 is adjusted to specific value using hydrochloric acid solution or sodium hydroxide solution.

6. Sodium formaldehyde sulfoxylate is added to the solution of step 5 processing temperature of 2° C. to 8° C. and stirred till clear solution is obtained.

7. Volume is made up with remaining water for injection.

8. Solution of step 7 is purged with nitrogen and subsequently filtered through 0.22 μm PVDF sterile filters.

9. This bulk solution is analyzed for impurity content using suitable HPLC method.

| Time | Impurity | 12 | 13 | 14 | 15 |
|---|---|---|---|---|---|
| Initial | TI | 0.91 | 0.86 | 0.76 | 0.75 |
| | MIUI | 0.21 | 0.20 | 0.19 | 0.15 |
| | Imp F | 0.10 | 0.10 | 0.08 | 0.07 |

*TI-Total Impurities
*MIUI-Maximum individual unknown impurity

From above data, it is evident that order of addition of drug and excipients does not have any impact on impurity profile of the product.

### Example 16-18: Liquid Pharmaceutical Formulation of Pemetrexed Disodium

| Ingredients | 16 | 17 | 18 |
|---|---|---|---|
| Pemetrexed disodium equivalent to | 40 | 40 | 40 |
| Pemetrexed (mg/ml) | | | |
| Mannitol (mg/ml) | 25 | 25 | 25 |
| Sodium formaldehyde sulfoxylate (mg/ml) | 0.20 | 0.10 | 0.10 |
| Disodium EDTA (mg/ml) | 0.25 | 0.25 | 0.50 |
| Water for Injection | 1 | 1 | 1 |
| | ml | ml | ml |

Manufacturing Process:

1. Disodium EDTA is added to 90% of required Water for injection and stirred till clear solution is obtained. Dissolved oxygen level is maintained below 2 ppm during bulk solution manufacturing.

2. Mannitol and Sodium formaldehyde sulfoxylate are added to the solution of step 1 under stirring and stirred till clear solution is obtained.

3. Pemetrexed disodium is added to the solution from above step and stirred till clear solution is obtained.

4. pH of the solution of step 3 is adjusted to 7 to 8 hydrochloric acid solution or sodium hydroxide solution and volume is made up with remaining water for injection

5. Solution of step 4 is purged with nitrogen and subsequently filtered through 0.22 μm sterile filters. Solution is stored under nitrogen blanket until filling is initiated.

6. The solution of step 5 is dispensed into depyrogenated USP type-I glass vials and stoppered in presence of nitrogen and sealed.

7. The solution filled vials are heat sterilized at 121° C.

8. Vials are initial and stored at 25±2° C. and 60% relative humidity (RH), or at 40±2° C. and 75% RH, for 3 months.

9. The contents of the initial and stored vials are analyzed for impurity content using suitable HPLC method. Data provided in below table.

| Time | Impurity | 16 | 17 | 18 |
|---|---|---|---|---|
| Initial (%) | TI | 0.30 | 0.30 | 0.15 |
| 3 Month 25° C./60% RH | TI | 1.07 | 0.78 | 0.84 |
| (%) | MIUI | 0.07 | 0.06 | 0.06 |
| | Imp F | 0.70 | 0.52 | 0.53 |
| 3 Month at 40° C./75% RH | TI | 1.54 | 1.14 | 1.11 |
| (%) | MIUI | 0.09 | 0.08 | 0.06 |
| | Imp F | 1.11 | 0.76 | 0.80 |

*TI-Total Impurities
*MIUI-Maximum individual unknown impurity

From above data, it is evident that formulation with sodium formaldehyde sulfoxylate and EDTA provides stable liquid pharmaceutical formulation of pemetrexed or pharmaceutically acceptable salt thereof.

### Example 19-21: Liquid Pharmaceutical Formulation of Pemetrexed Disodium

| Ingredients | 19 | 20 | 21 |
|---|---|---|---|
| Pemetrexed sisodium equivalent to | 40 | 40 | 40 |
| Pemetrexed (mg/ml) | | | |
| Mannitol(mg/ml) | 25 | 25 | 25 |
| Sodium formaldehyde sulfoxylate | 0.1 | 0.1 | 0.1 |
| (mg/ml) | | | |
| Water for Injection | 1 | 1 | 1 |
| | ml | ml | ml |
| pH | 7 | 7.75 | 8.5 |

Manufacturing Process:

1. Sodium formaldehyde sulfoxylate is added to 90% of required Water for injection and stirred till clear solution is obtained. Dissolved oxygen level is maintained below 2 ppm during bulk solution manufacturing.

2. Mannitol is added to the solution of step 1 under stirring and stirred till clear solution is obtained.

3. Pemetrexed disodium is added to the solution from above step and stirred till clear solution is obtained.

4. pH of the solution of step 3 is adjusted to specific value using hydrochloric acid solution or sodium hydroxide solution and volume is made up with remaining water for injection.

5. Solution of step 4 is purged with nitrogen and subsequently filtered through 0.22 μm sterile filters. Solution is stored under nitrogen blanket until filling is initiated.

6. The solution of step 5 is dispensed into depyrogenated USP type-I glass vials and stoppered in presence of nitrogen and sealed.

7. The solution filled vials are heat sterilized at 121° C.

8. Vials are stored at 25±2° C. and 60% relative humidity (RH), or at 40±2° C. and 75% RH, for 3 months.

9. The contents of the initial and stored vials are analyzed for impurity content using suitable HPLC method. Data provided in below table.

| Time | Impurity | 19 | 20 | 21 |
|---|---|---|---|---|
| Initial (%) | TI | 0.22 | 0.12 | 0.31 |
| 3 Month 25° C./60% RH | TI | 0.96 | 0.85 | 1.43 |
| (%) | MIUI | 0.10 | 0.08 | 0.10 |
| | Imp F | 0.53 | 0.51 | 0.48 |
| 3 Month at 40° C./75% RH | TI | 1.47 | Not available | 1.15 |
| (%) | MIUI | 0.06 | Not available | 0.06 |
| | Imp F | 0.99 | Not available | 0.86 |

*TI-Total Impurities
*MIUI-Maximum individual unknown impurity

### Example 22-26: Liquid Pharmaceutical Formulation of Pemetrexed Ditromethamine

| | Quantity per unit | | | | |
|---|---|---|---|---|---|
| Ingredients | 22 | 23 | 24 | 25 | 26 |
| Pemetrexed ditromethamine | 40 | 40 | 40 | 40 | 40 |
| equivalent to Pemetrexed | mg | mg | mg | mg | mg |
| Sodium metabisulfite | 3 | — | 3 | — | — |
| | mg | | mg | | |
| Sodium formaldehyde | — | 0.1 | 0.1 | 0.1 | 0.1 |
| sulfoxylate | | mg | mg | mg | |
| Disodium EDTA | 5 | 5 | 5 | 5 | — |
| | mg | mg | mg | mg | |
| Mannitol | — | — | — | 15 | — |
| | | | | mg | |
| Water for Injection | 1 | 1 | 1 | 1 | 1 |
| | ml | ml | ml | ml | ml |

1. Sodium metabisulfite or sodium formaldehyde sulfoxylate or both, as specified in above table is added to 90% of required Water for injection and stirred till clear solution is obtained. Dissolved oxygen level is maintained below 2 ppm during bulk solution manufacturing.

2. Disodium EDTA (for Example 29-32) is added to the solution of step 1 under stirring and stirred till clear solution is obtained. Mannitol is added to this solution under stirring and stirred till clear solution is obtained only in case of example 29.

3. Pemetrexed ditromethamine is added to the solution of step 2 and stirred till clear solution is obtained.

4. pH of the solution of step 3 is adjusted to 6.5 to 7.5 hydrochloric acid solution or tromethamine solution and volume is made up with remaining water for injection

5. Solution of step 4 is purged with nitrogen and subsequently filtered through 0.22 μm sterile filters. Solution is stored under nitrogen blanket until filling is initiated.

6. The solution of step 6 is dispensed into depyrogenated USP type-I glass vials and stoppered in presence of nitrogen and sealed.

7. The solution filled vials are heat sterilized at 121° C.

### Example 27: Liquid Pharmaceutical Formulation of Pemetrexed Ditromethamine

| Ingredients | Quantity per unit | |
|---|---|---|
| Pemetrexed ditromethamine | 40 | mg/ml |
| equivalent to Pemetrexed | | |
| Sodium formaldehyde sulfoxylate | 0.1 | mg/mL |
| Disodium EDTA | 0.25 | mg/ml |
| Water for Injection | 1 | mL |

1. Water for injection is cooled to around 40° C. under nitrogen purging and Disodium EDTA is added under stirring. Dissolved oxygen level is maintained below 2 ppm during bulk solution manufacturing. Once clear solution is attained, solution was cooled to around 25° C.

2. Pemetrexed ditromethamine is added under stirring and dissolved under nitrogen purging.

3. Once clear solution is achieved sodium formaldehyde sulfoxylate was added and dissolved while nitrogen purging, pH is adjusted in between 7.0 to 8.5.

4. Solution of step 4 is purged with nitrogen and subsequently filtered through 0.22 μm PVDF sterile filters. Solution is stored under nitrogen blanket until filling is initiated.

5. The solution of step 5 is dispensed into depyrogenated USP type-I glass vials and stoppered in presence of nitrogen and sealed.

6. The solution filled vials are heat sterilized at 121° C.

1. A stable liquid pharmaceutical formulation of pemetrexed or pharmaceutically acceptable salts thereof comprises at least one stabilizing agent selected from the group consisting of sodium formaldehyde sulfoxylate, EDTA and derivatives and mixtures thereof.

2. A stable liquid pharmaceutical formulation of claim 1, wherein the pharmaceutically acceptable salt of pemetrexed is pemetrexed disodium.

3. A stable liquid pharmaceutical formulation of claim 1, wherein the formulation comprises from about 5% to about 80% of pemetrexed or pharmaceutically acceptable salts thereof.

4. A stable liquid pharmaceutical formulation of claim 3, wherein the formulation comprises from about 20% to about 50% of pemetrexed or pharmaceutically acceptable salts thereof.

5. A stable liquid pharmaceutical formulation of claim 1, wherein the formulation comprises sodium formaldehyde sulfoxylate from about 0.01 mg/ml to about 3 mg/ml.

6. A stable liquid pharmaceutical formulation of claim 5, wherein the formulation comprises sodium formaldehyde sulfoxylate from about 0.01 mg/ml to about 2 mg/ml.

7. A stable liquid pharmaceutical formulation of claim 1, wherein the formulation comprises EDTA or derivatives thereof from about 0.01 mg/ml to about 5 mg/ml.

8. A stable liquid pharmaceutical formulation of claim 7, wherein the formulation comprises EDTA or derivatives thereof from about 0.01 mg/ml to about 3 mg/ml.

9. A stable liquid pharmaceutical formulation of claim 1, wherein the formulation comprising pemetrexed is in the form of ready-to-use solution or concentrate for further dilution.

10. A stable liquid pharmaceutical formulation of claim 1, wherein the formulation comprises not more than about 3% of total impurities.

11. A stable liquid pharmaceutical formulation of claim 10, wherein the formulation comprises not more than about 3% of total impurities, not more than about 0.5% of maximum individual unknown impurity, not more than about 2% of impurity F.

12. An impurity F as per claim 11, having following structure:

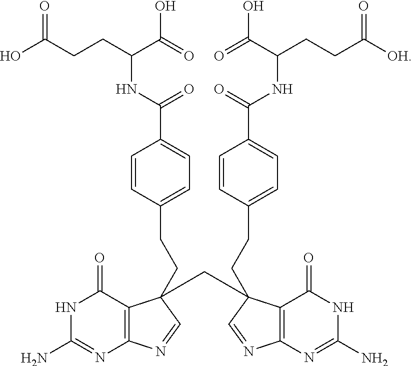 [SMILES: Nc1nc2c(c(=O)[nH]1)C(CCc1ccc(C(=O)NC(CCC(=O)O)C(=O)O)cc1)(CC1(CCc3ccc(C(=O)NC(CCC(=O)O)C(=O)O)cc3)C=Nc3nc(N)[nH]c(=O)c31)C=N2]

13. A stable liquid pharmaceutical formulation of claim 11, wherein the formulation comprises not more than about 2% of total impurities.

14. A stable liquid pharmaceutical formulation of claim 11, wherein the formulation comprises not more than about 0.2% of maximum individual unknown impurity.

15. A stable liquid pharmaceutical formulation of claim 11, wherein the formulation comprises not more than about 1% of impurity F.

16. A stable liquid pharmaceutical formulation of claim 1, wherein the formulation is prepared by process comprising steps of:
(1) Purging nitrogen to reduce dissolved oxygen of solvent system below 2 ppm.
(2) Dissolving pemetrexed or pharmaceutically acceptable salt thereof in solvent system.
(3) Dissolving stabilizing agent, selected from the group consisting of sodium formaldehyde sulfoxylate, disodium edetate and mixtures thereof, in solvent system.
(4) Adjusting the pH of the solution from about pH 6 to about 8.5.
(5) Sterilizing using 0.22 μm sterile filters.
(6) Filling and sealing in suitable vial.

17. A process of claim 18, comprising steps of:
(1) Purging nitrogen to reduce dissolved oxygen of solvent system below 2 ppm.
(2) Dissolving pemetrexed or pharmaceutically acceptable salt thereof at a concentration from about 20 mg/ml to about 50 mg/ml in solvent system.
(3) Dissolving sodium formaldehyde sulfoxylate at a concentration from about 0.01 mg/ml to about 5 mg/ml in solvent system.
(4) Adjusting the pH of the solution from about pH 6 to about 8.5.
(5) Sterilizing using 0.22 μm sterile filters.
(6) Filling and sealing in suitable vial.

18. A process of claim 19, comprising steps of:
(1) Purging nitrogen to reduce dissolved oxygen of solvent system below 2 ppm.
(2) Dissolving pemetrexed or pharmaceutically acceptable salt thereof at a concentration from about 20 mg/ml to about 50 mg/ml in water at processing temperature in a range from about 2° C. to about 8° C.
(3) Dissolving sodium formaldehyde sulfoxylate at a concentration from about 0.01 mg/ml to about 5 mg/ml in water at processing temperature in a range from about 2° C. to about 8° C.
(4) Adjusting the pH of the solution from about pH 7 to about 8.
(5) Sterilizing using 0.22 μm sterile filters.
(6) Filling and sealing in suitable vial.
